(19) United States
(12) Patent Application Publication
(10) Pub. No.: US 2018/0090961 A1
(43) Pub. Date: Mar. 29, 2018

(54) BATTERY FOR MEDICAL DEVICES WITH REMOVABLE POWER CARTRIDGES

(71) Applicants: Krystyna Namolovan, Toronto (CA); Roman Korytski, Toronto (CA)

(72) Inventors: Krystyna Namolovan, Toronto (CA); Roman Korytski, Toronto (CA)

(21) Appl. No.: 15/717,878

(22) Filed: Sep. 27, 2017

Related U.S. Application Data
(60) Provisional application No. 62/400,292, filed on Sep. 27, 2016.

(51) Int. Cl. H02J 7/00; A61M 16/00

(52) U.S. Cl. CPC H02J 7/0054; A61M 2205/8206; A61M 16/021; H02J 7/0055

(57) ABSTRACT

System for charging portable medical devices, the system comprising a battery, which comprises a case and a power cartridge. The case comprises a circuit board, a plug, and a display unit. The power cartridge comprises at least one slot for holding an interchangeable, rechargeable battery. The plug is configured to allow for charging of a portable medical device. And the display unit is configured to display how much charging energy remains in the battery.

### CROSS-REFERENCE TO RELATED APPLICATIONS

This application claims the benefit of U.S. Provisional Patent Application No. 62/400,292 filed on Sep. 27, 2016, the contents of which are hereby incorporated by reference.

### FIELD OF THE INVENTION

The present invention relates generally to apparatus and methods for powering and operating medical devices, and, more particularly, portable oxygen concentrators.

### BACKGROUND

There is a burgeoning need for home and ambulatory oxygen. Supplemental oxygen is necessary for patients suffering from lung disorders; for example, pulmonary fibrosis, sarcoidosis, or occupational lung disease. For such patients, oxygen therapy is an increasingly beneficial, life-giving development. While not a cure for lung disease, supplemental oxygen increases blood oxygenation, which reverses hypoxemia. This therapy prevents long-term effects of oxygen deficiency on organ systems—in particular, the heart, brain and kidneys.

Stationary sources of oxygen are available, e.g., oxygen lines in hospitals or other facilities, that may provide oxygen to patients. To allow some mobility, cylinders of pure and/or concentrated oxygen can be provided that a patient may carry or otherwise take with them, e.g., on pull-along carts. Such cylinders, however, have limited volume and are large and heavy, limiting the patient's mobility.

Oxygen treatment is also prescribed for Chronic Obstructive Pulmonary Disease (COPD), which afflicts about six-hundred million people in the U.S., and for other ailments that weaken the respiratory system, such as heart disease and AIDS. Supplemental oxygen therapy is also prescribed for asthma and emphysema.

Portable devices, which concentrate oxygen from ambient air, are often used to provide supplemental oxygen.

Respiratory oxygen usage rates typically range up to 3 LPM (liters per minute at 22° C. and 1 atm pressure) for ambulatory patients with relatively low oxygen requirements, up to 5 LPM for patients with more serious respiratory problems and possibly limited mobility, and in certain cases up to 10 LPM for those with the most serious respiratory problems and more limited mobility. A patient initially may require a higher oxygen supply rate during an illness and later may require less oxygen as recovery is achieved. Alternatively, a patient may require increasing oxygen rates as a chronic condition worsens. A conserver may be used to provide oxygen flow only when the patient inhales, thereby reducing the amount of oxygen required by eliminating the supply of oxygen that is wasted when the patient exhales.

A portable oxygen concentrator (POC) is a portable device used to provide oxygen therapy to patients at substantially higher oxygen concentrations than the levels of ambient air. It is very similar to a home oxygen concentrator, but is smaller in size and more mobile. The portable oxygen concentrator makes it easy for patients to travel freely; they are often small enough to fit in a car and many concentrators are now FAA-approved.

Portable oxygen concentrators (POCs) often are preferred over liquid or compressed oxygen supply systems in home and residential settings, and small air separation devices for these applications are being developed by numerous vendors in the home health care field. Patients typically are encouraged to be ambulatory whenever possible to increase the effectiveness of oxygen therapy and improve their overall health. The portability of a portable oxygen concentrator therefore is an important feature allowing the patient to move about easily and comfortably. In order to maximize portability and ease of use, the medical oxygen concentrator must be designed to have minimum weight and compact dimensions. Patient ambulation time can be maximized by the use of a conserver.

Portable oxygen concentrators have been around for decades, but the older versions were bulky, unreliable, and were not permitted on airplanes. Since 2000, manufacturers have improved their reliability and they now produce anywhere between one to six liters per minute (LPM) of oxygen. The portable concentrators may plug directly into a regular house outlet for charging at home or hotel, but they come with a power adapter that can usually be plugged into a vehicle DC adapter. Many have the ability to operate from the battery power as well for either ambulatory use, or away from a power source, or on an airplane.

Portable oxygen therapy allows oxygen therapy patients to maintain their mobility and independence throughout their day-to-day activities. Portable oxygen concentrators have many benefits. They allow patients to utilize oxygen therapy 24/7, which helps increase survival. Portable oxygen concentrators help improve exercise tolerance, as supplemental oxygen during exercise helps users exercise longer. POCs help increase stamina throughout day-to-day activities. POCs allow for freedom to travel lightly and easily. There is no need to carry around heavy oxygen tanks, which are cumbersome, heavy, and dangerous (explosive properties).

POCs operate on the same principle as a home domestic concentrator, operating through a series of cycles. Air at barometric pressure contains 21% oxygen combined with nitrogen and a mixture of other gases. A miniaturized air compressor inside the machine will pressurize this air through a system of chemical filters known as a molecular sieve. This filter is made up of silicate granules called Zeolite, which sieves the nitrogen out of the air, concentrating the oxygen. Part of the oxygen produced is delivered to the patient; part is fed back into the sieves to clear them of the accumulated nitrogen, preparing them for the next cycle. Through this process, the system is capable of producing medical grade oxygen of up to 90% consistently. The latest models can be powered from mains electricity supply, 12V DC (car/boat etc.), and battery packs making the patient free from relying on using cylinders and other current solutions that put a restriction on time, weight, and size.

POCs can be divided into two categories: on-demand/intermittent flow (IF) and continuous flow (CF). Most of the current portable oxygen concentrator systems provide oxygen on a pulse (on-demand) delivery in order to maximize the purity of the oxygen. The system supplies a high concentration of oxygen and is used with a nasal cannula to channel oxygen from the concentrator to the patient.

On-demand (also called intermittent-flow or pulse-dose) POCs are the smallest, often no bigger than a briefcase or picnic cooler and weighing in the range of 2.8 to 9.9 pounds (1.3 to 4.5 kg). These deliver oxygen only when patients inhale, avoiding the waste of oxygen during exhalation. Their ability to conserve oxygen is key to keeping the units so compact without sacrificing the duration of oxygen supply. Sleek, slim exterior cases, some with form-fitted carrying bags, optimize the flexibility to take these units almost anywhere—even to high altitudes over 10,000 feet—as long as there's sufficient battery run time until the next opportunity to recharge.

Although each category includes multiple brands with varied characteristics, the most important consideration for any POC is its ability to supply adequate supplementary oxygen to relieve hypoxia (oxygen deficiency) during normal activities of daily living. With continuous-flow, oxygen delivery is measured in LPM (liters per minute). The continuous flow (CF) units weigh between 10 and 20 pounds (4.5 to 9.0 kg). With on-demand or pulse-flow, delivery is measured by the size (in milliliters) of the “bolus” of oxygen per breath, referring to a burst of oxygen released at the instant of inhalation. Other important variables include maximum oxygen purity (oxygen percentage), the number and increment of settings for adjusting oxygen flow, and battery capacity (or number of add-on batteries) and power cord options for recharging.

The Federal Aviation Administration (FAA) regulations concerning respiratory assistive devices on aircraft are codified in the Department of Transportation (DOT) Final Rule “Nondiscrimination on the Basis of Disability in Air Travel” and 14 CFR 382.133. Under 14 CFR 382.133(f)(2), an airline may require an individual to bring an adequate number of fully charged batteries onboard, based on the battery manufacturer's estimate of the hours of battery life while the device is in use and the information provided in the physician's statement, to power the device for not less than 150% of the expected maximum flight duration. However, US DOT regulations, 49 CFR 175.10(a)(18), state that “each installed or spare lithium battery must not exceed the following (i) For a lithium metal battery, a lithium content of not more than 2 grams per battery; or (ii) For a lithium ion battery, the Watt-hour rating must not exceed 100 Wh. With the approval of the operator, portable electronic devices may contain lithium ion batteries exceeding 100 Wh, but not exceeding 160 Wh and no more than two individually protected lithium ion batteries each exceeding 100 Wh, but not exceeding 160 Wh, may be carried per person as spare batteries in carry-on baggage.” Based on the DOT rules that limit battery energy and usage, the use of POCs is severely limited on airplanes.

Current batteries that are available on the market for medical devices are either under 100 wh, but provide insufficient run time for a medical device for 150% of the expected maximum flight duration, or are over 100 Wh but are refused by airlines to be used on board, causing patients big inconveniences. Oftentimes, patients are required to leave their POC devices behind.

Accordingly, there is a need for a more efficient method of powering POCs on airplanes within the DOT requirements.

### BRIEF DESCRIPTION OF THE DRAWINGS

A set of hand drawings and photos of a mockup of the present invention are provided herewith for display purposes. One of ordinary skill in the art would appreciate that these are for illustrative purposes only and that there could be many variations and embodiments of the present invention, formed in a variety of shapes and sizes. These illustrations, along with the detailed description below, would enable one of ordinary skill in the art to understand and practice the invention:

FIG. 1 is an example of the front and back of the battery with removable power cartridges;

FIG. 2 is an example of the rear of the battery;

FIG. 3 is an example of the front of the battery;

FIG. 4 is an example of the battery with one of the removable power cartridges removed;

FIG. 5 is a first exploded view of the battery;

FIG. 6 is a second exploded view of the battery; and

FIGS. 7A and 7B illustrate the connection between the removable power cartridge and the case.

### DETAILED DESCRIPTION OF CERTAIN INVENTIVE EMBODIMENTS

Embodiments of this application relate to providing power to POC devices. Batteries may be used to provide power to small medical devices such as Positive Airway Pressure (PAP) machines and portable oxygen concentrators (POCs). Embodiments of this application may be used to provide power to the POCs devices in order to make them portable. POCs are equipped with original manufacturer internal batteries. Unfortunately, the original manufacturer internal batteries last very short time. Embodiments of the current invention may be used as an external battery that will extend the portability of POCs and give a patient/user more time to remain outside their home.

According to an embodiment of the invention, a battery may be comprised of two parts: (1) a case; (2) and at least one power cartridge. The case may have the motherboard (circuit board), which contains the “brain” of the device. The case may comprise ports to plug in the medical device, ports to plug in the charger to charge the battery, a display with the amount of charge left in the cartridges, and extra optional features such as Universal Serial Bus (USB) ports and a flash light. In one embodiment, the user may electrically connect two or more cases together.

Each case may hold one or more power cartridges. In one compact embodiment of the invention, the battery may comprise only one power cartridge. In another embodiment of the invention, the case may comprise two or more power cartridges. A power cartridge may comprise non-chargeable or chargeable batteries such as lead—acid, nickel cadmium (NiCd), nickel metal hydride (NiMH), lithium ion (Li-ion), and lithium ion polymer (Li-ion polymer). The power cartridge may further comprise power cells and a small circuit board that allows the patient/user to see the amount of charge left in a cartridge. The cartridges may be plugged into the case. The patient may use one case and as many cartridges as they want. Once a cartridge drains out of power, the patient may replace it with another one. Due the FAA regulations, the cartridges may have a capacity of 100 Wh or less. US DOT regulations, 49 CFR 175.10(a)(18), state that “each installed or spare lithium battery must not exceed the following (i) For a lithium metal battery, a lithium content of not more than 2 grams per battery; or (ii) For a lithium ion battery, the Watt-hour rating must not exceed 100 Wh. With the approval of the operator, portable electronic devices may contain lithium ion batteries exceeding 100 Wh, but not exceeding 160 Wh and no more than two individually protected lithium ion batteries each exceeding 100 Wh, but not exceeding 160 Wh, may be carried per person as spare batteries in carry-on baggage.” The Watt-hour capacity of the cartridge may be higher than 100Wh.

Each power cartridge may comprise one or more rechargeable batteries. In at least one embodiment of the invention, the patient may effectively bring on board of the aircraft a 300 Wh battery, for example, that meets the Department of Transportation (DOT) requirements. Typically, a 300 wh battery is not allowed on a plane. But this embodiment circumvents the rules because it uses three individual 100 wh batteries, which are within the DOT energy limits. To be safe, the individual batteries may be 98 Wh or 99 Wh.

The battery described herein may also be used as a Continuous Positive Airway Pressure (CPAP) battery for sleep apnea treatment machines.

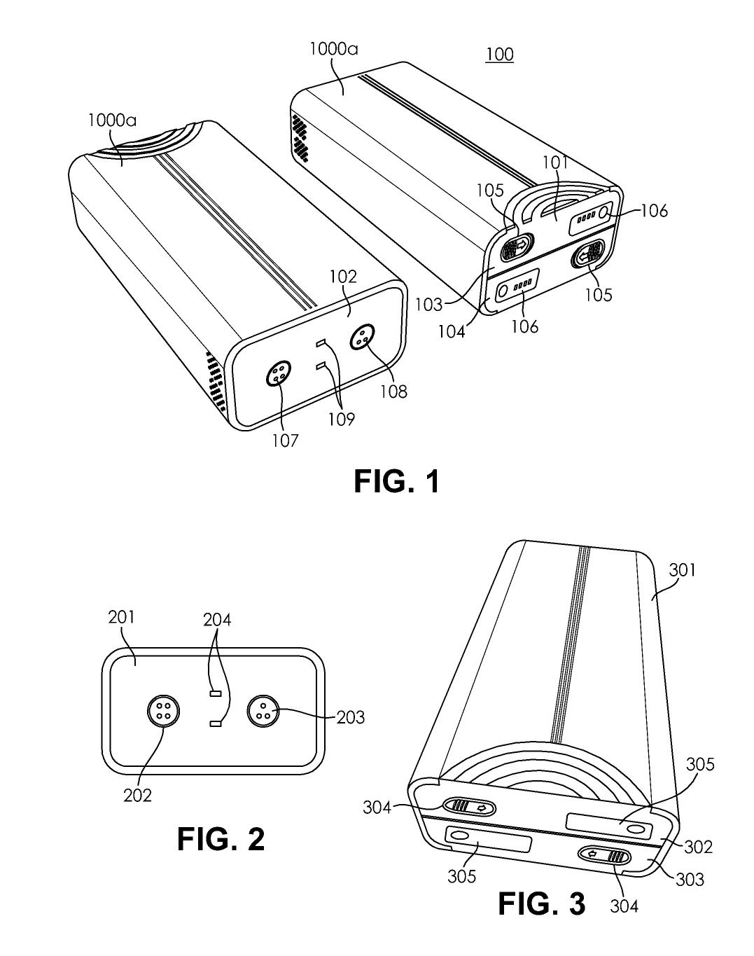

FIG. 1 is an example of the front and back of the battery with removable power cartridges. The battery 100 may include a case 100a, a front 101, and a back 102. The front of the battery 101 may include a first removable power cartridge 103 and a second removable power cartridge 104. The first and second removable power cartridges 103 and 104 include a release button 105. The release button 105 may be a slider button. The first and second removable power cartridges 103 and 104 may include a charge indicator 106. The rear of the battery 102 may include plugs (a charge port 107 and a DC out port 108). The rear of the battery 102 may also include cell 1 and cell 2 indicators 109. The cell 1 and cell 2 indicators 109 may be used to indicate the presence of a cell inside of the battery.

FIG. 2 is an example of the rear of the battery. The rear of the battery 201 may include plugs (a charge port 202 and a DC out port 203). The rear of the battery 201 may also include cell 1 and cell 2 indicators 204. The cell 1 and cell 2 indicators 204 may be used to indicate the presence of a cell inside of the battery. When there is a cell present inside of the battery one of the cell indicators 204 will light up.

FIG. 3 is an example of the front of the battery. The front of the battery 301 may include a first removable power cartridge 302 and a second removable power cartridge 303. The first and second removable power cartridges 302 and 303 include a release button 304. The release button 304 may be a slider button. The release button 304 may be slid to allow at least one of the first and second removable power cartridges 303 and 303 from the battery. The first and second cartridges 302 and 303 may include a charge indicator 305. The charge indicator 305 indicates to a user the level of charge. This way the user is able to determine when the battery is fully charges. Each of the first and second removable power cartridges are separately removable.

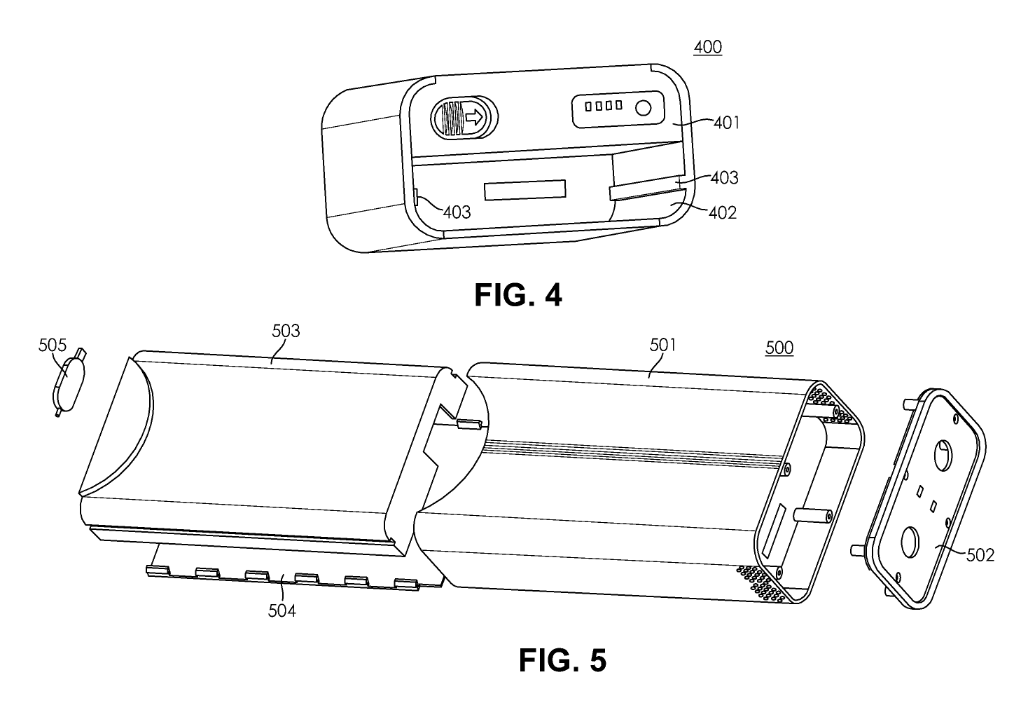

FIG. 4 is an example of the battery with one of the removable power cartridges removed. The battery 400 illustrated in FIG. 4 shows one of the removable power cartridges removed from the battery case 401. An empty hole 402 in the battery case 401 includes grooves 403 that allow a removable power cartridge to slide in and out of the battery case 401.

FIG. 5 is a first exploded view of the battery. The battery 500 includes a case 501, an end cap 502, a removable power cartridge 503, a removable power cartridge cover 504, and a button 505. The end cap 502 attaches to the case 501. The removable power cartridge cover 504 attaches to the removable power cartridge 503 to make one piece that fits into the case 501. The button 505 attaches to the end of the removable power cartridge 503. The button 505 may be used to release the removable power cartridge 503 from the case 501.

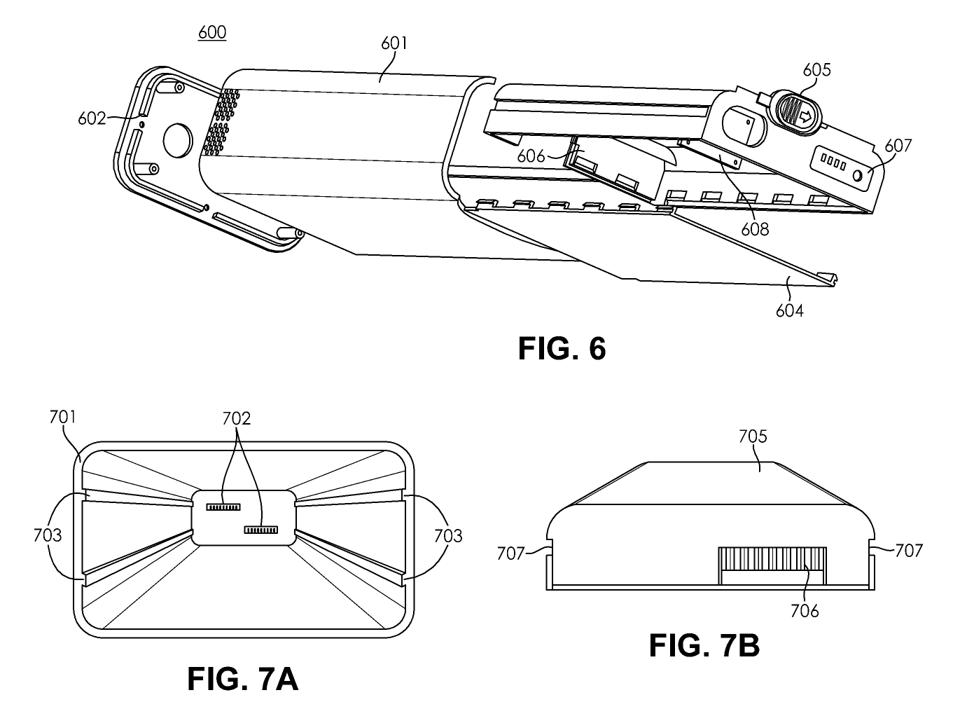

FIG. 6 is a second exploded view of the battery. The battery 600 includes a case 601, an end cap 602, a removable power cartridge 603, a removable power cartridge cover 604, and a button 605. The removable power cartridge 603 includes a bottom 606. The bottom 606 may be comprised of conductive material that allows the batteries inside of the removable power cartridge to connect to the case 501 for charging. The button 605 may be used to release the removable power cartridge 603 from the case 601. The removable power cartridge 603 may include a charge indicator 607. The removable power cartridge 603 may have a plate 608 on the inside of the button 605.

FIGS. 7A and 7B illustrate the connection between the removable power cartridge and the case. FIG. 7A illustrates the inside of the case 701. The inside of the case 701 may include at least one charging connection 702. The at least one charging connection 702 may be a male connection. The case 701 may include grooves 703. The grooves 703 may be used for sliding at least one removable power cartridge into the case 701.

FIG. 7B illustrates the bottom of the removable power cartridge 705. The bottom of the removable power cartridge 705 may include a charging connection 706. The charging connection 706 may be a female connection. The removable power cartridge 705 may include indentations 707 for sliding the removable power cartridge 705 into the grooves of the case.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

What is claimed is:

1. A system for charging portable medical devices, the system comprising:
a battery, comprising a case and at least one removable power cartridge;
wherein said case comprises a first charging connection, a plug, and a display;
wherein said removable power cartridge comprises at least one slot for holding an interchangeable, rechargeable battery;
wherein the plug is configured to allow for charging of the portable medical device;
wherein the display is configured to indicate how much charging energy remains in the battery.

2. The system of claim 1, wherein the removable power cartridge further comprises a second charging connection to connect to the first charging connection.

3. The system of claim 1, wherein the removable power cartridge further comprises a release button.

4. The system of claim 1, wherein the removable power cartridge further comprises a charge indicator.

5. The system of claim 1, wherein the removable power cartridge further comprises at least one rechargeable battery.

6. The system of claim 5, wherein the at least one battery is a lithium ion battery.

7. The system of claim 1, wherein the case further comprises at least one Universal Serial Bus (USB) port.

8. The system of claim 1, wherein the care further comprises a flash light.

9. The system of claim 1, wherein the battery is used for a Positive Airway Pressure (PAP) machine.

10. The system of claim 1, wherein the battery is used for a portable oxygen concentrator (POC).

11. The system of claim 1, wherein the battery is used as a Continuous Positive Airway Pressure (CPAP) battery for a sleep apnea machine.
